SEQUENCE LISTING (3 sequences)

SEQ ID NO: 1 - DNA, 743 bp, Pseudomonas aeruginosa
ccatgggcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatggtaccggcagcagccatcatcatcatcatcatggcagcggtctggtgccgcgtggcagcgcgagcatgagcgatagcgaggtgaaccaggaagcgaagccggaggtcaagccggaggtcaagccggagacgcacatcaacctgaaggtcagcgatggcagctctgagattttcttcaagatcaagaagaccacgccgctgcgtcgtctgatggaggcgttcgctaagcgtcaaggcaaggagatggacagccttcgcttcctgtacgatggcatccgcattcaagctgatcagactccggaggacctggatatggaggacaacgacatcatcgaagctcatcgtgagcagatcggaggccagcattggagctattggctgcgtccgggccatatg

SEQ ID NO: 2 - DNA, 1443 bp, Pseudomonas aeruginosa
atgggtaccggcagcagccatcatcatcatcatcatggcagcggtctggtgccgcgtggcagcgcgagcatgagcgatagcgaggtgaaccaggaagcgaagccggaggtcaagccggaggtcaagccggagacgcacatcaacctgaaggtcagcgatggcagctctgagattttcttcaagatcaagaagaccacgccgctgcgtcgtctgatggaggcgttcgctaagcgtcaaggcaaggagatggacagccttcgcttcctgtacgatggcatccgcattcaagctgatcagactccggaggacctggatatggaggacaacgacatcatcgaagctcatcgtgagcagatcggaggccagcattggagctattggctgcgtccgggccatatggcggaagaaggtggctctctggcagcgctgaccgcccatcaggcatgtcatctgccgctggaaaccttcacccgtcaccgccagccgcgtggttgggaacaactggaacagtgcggttacccggtacagcgtctggtcgctctgtacctggcagctcgtctgtcctggaaccaggttgatcaggttatccgtaacgcgctggcgtctccgggttctggtggtgatctgggcgaagcaatccgtgagcagccggagcaggcccgcctggcactgactctggcggctgccgaatccgaacgtttcgttcgccagggcaccggcaatgatgaagcgggtgctgcaaacggtccagcggatagcggtgatgcactgctggaacgcaactacccgaccggtgcggaatttctgggtgacggtggtgacgtgtcttttagcactcgtggtactcaaaactggacggtagaacgtctgctgcaggcgcaccgtcagctggaagagcgtggttacgttttcgttggctaccacggcactttcctggaagcagcgcagtctatcgtgtttggcggcgttcgtgctcgtagccaggacctggatgcgatttggcgtggtttctatattgctggcgacccggctctggcttatggttatgcgcaggatcaagagcctgacgcccgtggccgtattcgcaacggcgcgctgctgcgcgtatacgtgccgcgctccagcctgccgggcttctatcgtaccagcctgaccctggctgcgccggaagccgctggtgaggtggaacgcctgatcggccacccgctgccactgcgcctggacgccatcactggcccagaagaagaaggcggtcgtctggagacgatcctgggctggcctctggccgaacgtaccgttgtcatcccgtccgcaattccgaccgatccgcgcaacgtgggtggcgatctggacccgtcctctatcccagacaaggaacaagctatctctgctctgcctgactacgcatcccagccgggtaaaccgccgaaagacgaactgtaa

SEQ ID NO: 3 - protein, 359 aa, Pseudomonas aeruginosa
QHWSYWLRPGHMAEEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

(19) United States
(12) Patent Application Publication
(10) Pub. No.: US 2016/0222362 A1
(43) Pub. Date: Aug. 4, 2016

(54) Target-Specific Double-Mutant Fusion Protein and Preparation Process Therefor

(71) Applicant: BEIJING BOAUTAI BIOTECHNOLOGY CO., LTD., Beijing (CN)

(72) Inventors: Junying Zhang, Hengshui, Hebei (CN); Xiaoyun Hou, Changchun (CN)

(21) Appl. No.: 14/889,396

(22) PCT Filed: Mar. 31, 2014
(86) PCT No.: PCT/CN2014/000353

§ 371 Date: Nov. 5, 2015

(30) Foreign Application Priority Data
Mar. 24, 2014 (CN) 201410108673.9

(51) Int. Cl. C12N 9/10; C07K 7/23

(52) U.S. Cl. CPC C12N 9/1077; C07K 7/23; C07K 2319/55; A61K 38/00

(57) ABSTRACT

Disclosed are a fusion protein of a human gonadotrophin-releasing hormone mutant (mGnRH) and a pseudomonas aeruginosa exotoxin A mutant (PE38m4a), and a preparation method therefor. The fusion protein can be used for preparing an antitumor drug.

### TECHNICAL FIELD

The invention belongs to the field of molecular biology, and the invention generally pertains to the new preparation technology of fusion proteins with the specific cytotoxicity of a target cell. More specifically, the invention pertains to the preparation technology for gene reconstruction and purification for the tumor specific drugs—human gonadotropin releasing hormone mutants (mGnRH) and recombinant Pseudomonas aeruginosa exotoxin A truncated fragment or its mutant (PE38m4a) fusion protein.

### BACKGROUND TECHNOLOGY

In order to improve the selective killing effect of anti tumor drugs on tumor cells, it has constructed and expressed in the Chinese patent CN200810051112.4 the double-mutant fusion protein which takes the mutated GnRH as oriented, with Pseudomonas aeruginosa exotoxin A truncated and mutated at the C-terminal as cytotoxic agents, and its basic principle is that in the process of the occurrence and development of tumor, many tumor cells will over express gonadotropin releasing hormone receptor protein (GnRHR) on their surface, therefore, we can connect certain cytotoxic agents (such as Pseudomonas exotoxin A (PE), diphtheria toxin, cholera toxin, staphylococcus endotoxin and ricin, etc.) to the GnRH molecules which serve as the directing agent, to produce hybrid molecules that have both tumor cell directing function and cytotoxic activity. These hybrid molecules, with the aid of the directing capacity of the target tumor cells, will lead the entire molecule to the target cells and kill the target cells by its toxin part.

As the cytotoxic agent, Pseudomonas aeruginosa exotoxin A (PEA) (see U.S. Pat. No. 4,545,985 and Chinese patent application CN200810051112.4) is a single chain polypeptide composed of 613 amino acids. X-ray crystallographic studies and mutation analysis of PEA molecule show that the PEA molecule includes three structural domains related to production of cytotoxicity: the amino terminal cell receptor binding domain which is responsible for binding with sensitive cell (domain I); the intermediate transposition domain responsible for translocating toxin molecules into the cytosol (domain II), and the carboxyl terminal enzymatic activity domain responsible for inactivation of proteins and eventually leading to death of the cell (domain III). The domain I includes the Ia region (amino acids 1-252) where mediating cells are bound, and the Ib region (amino acid 365-399) the function of which has not been defined. Biochemistry or recombinant DNA technology can be used to modify the PEA molecular, so as to prepare various modified PEA fragments in PEA molecule where one or more amino acids is are missing or substituted, for example, generally the PE-A proteins the Ia region in the PE molecular of which is deleted, containing only the enzymatic and transposition region, with a molecular weight of about 40 kDa are called PE40. Now it has been found that after deleting the Ia region and majority of the Ib (amino acids 365-380) region of PE, that is, the PE38 toxin molecule, the molecule retains its specific cytotoxicity, but the non-specific toxicity has been reduced (for example, refer to Hwang et al., Cell 48:129-136, 1987; U.S. Pat. No. 4,892,827, and the European patent 0261671). At the same time, if the C terminal amino acid RKEDL in PE38 molecule is mutated into the KDEL which closely binds with endoplasmic reticulum and Golgi apparatus, the cellular activity can be increased by more than 10 times.

A number of existing technical documents have described the method of the fusion of PEA with various growth factors, antibodies, hormones, or CD4, to produce a hybrid protein that can selectively lead to and kill the target cells that have different cell membrane proteins (receptors or antigens). The U.S. Pat. No. 5,428,143 discloses the hybrid protein used for the selective killing of HIV infected cells and the construction of the chimeric gene that encodes this hybrid protein. The chimeric gene therein described consists of the human CD4 which contains the HIV binding site and the protein (PE40) that can kill the toxicity of HIV infected cells.

As an existing technology more relevant to this the invention, the PCT international patent application WO93/15751 discloses the chimeric protein molecule produced by directly coupling the gonadotropin releasing hormone (GnRH) peptide to the Pseudomonas aeruginosa exotoxin molecules. It is said that the use of such chimeric molecule may lead to the destruction of cells carrying the GnRH receptor in the pituitary, accompanied with the reduction of sex hormone secretion, therefore, it is expected to be used in animal sterilization and suppression of the proliferation of steroid hormone related tumors.

What is directly related to this invention is that the Chinese patent CN200810051112.4 held by the applicant provides a kind of chimeric toxin created by the fusion of the mutated human gonadotrophin and the recombinant pseudomonas aeruginosa exotoxin A mutant, characterized by that the mutated human gonadotrophin part described, serves as the directing agent and can bind with the peripheral gonadal hormone reactive or non reactive tumor cells specificity that have corresponding hormone receptor on the surface, and when internalized into tumor cells, the pseudomonas aeruginosa exotoxin part described can effectively kill the tumor cells as the cytotoxic agent. There directing hormone therein described is the mutated gonadotrophin releasing hormone (mGnRH), and the recombinant pseudomonas aeruginosa exotoxin there in described is PE38KDEL (PE38 m4a).

### CONTENTS OF THE INVENTION

According to the Chinese patent CN200810051112.4 held by the applicant, the invention has been further invented and innovated by in the aspects of specific gene engineering operation and product purification, which makes its structure more faithful to the natural structure of GnRH, and makes the purification process more simple and economical and practical, with significant increase in the yield.

According to a preferred implementation plan of the invention, the mGnRH described therein is connected into the PE molecule at the position equivalent to the Ia region cells in the form of artificial in-vitro gene synthesis.

According to a preferred implementation plan of the invention, the mGnRH described therein is synthesized in genetic form dominated by Escherichia coli preferred codon.

Another purpose of the invention is to provide the aforementioned mGnRH-PE38 m4a chimeric toxin which contains an effective amount of cytotoxicity, and one or more pharmaceutically acceptable vector or excipient pharmaceutical compositions.

Another purpose of the invention involves the application of the above-mentioned pharmaceutical compositions in treatment of various tumors associated with the gonadotropin receptor.

A preferred scheme of the invention is to splice the transsulfurase A which is conductive to the formation of disulfide bond with the SUMO protease recognition substrate sequence and the mGnRH-PE38 m4a gene in Chinese patent CN200810051112.4 in proper order, to form the fusion expression and remove the Met-Gly amino acid which originally serves as the enzyme digestion connection sequence of the N terminal of mGnRH-PE38 m4a.

Another preferred scheme of the invention is to add histidine tag His6 with the metal chelating medium affinity at the N terminal of the SUMO protease recognition substrate sequence.

Another preferred scheme of the invention is to purify the metal chelating medium by using 20 mM imidazole, to get the fusion protein with a purity of 90% by one step.

Another preferred scheme of the invention is to make use of the high specificity and highly active SUMO protease to conduct enzyme digestion of the purified fusion protein, to enable the label part to isolate from the mGnRH-PE38m4 target protein, and conduct metal chelating medium purification with the presence of 20 mM imidazole, to get the mGnRH-PE38m4a target protein with a purity of 95% in the form of spreading.

Another preferred scheme of the invention is that the highly purified target proteins, in the presence of a variety of protective agents, are made into pharmaceutical compositions with anti-tumor abilities.

The term “directing agent” used in this text refers to molecules or ligands that can bind only to the receptors or antigens specificity on the surface the target cell to be killed. The “directing agent” sometimes is referred to as the “recognition molecule” or the “ligand binding agent”. Examples of such recognition molecules are the antibodies or their fragments that can be identify the target cells, and the growth factors, cytokines, cytokines, antigens, and hormones that can bind with the molecular specificity on target cells. According to the preferred scheme of the invention, the directing agent described is the gonadal hormone (mGnRH) of small molecule mutation.

GnRH was isolated and purified from the animal by Schally and others in 1971, and was synthesized after the structure of it was described, and for this it won the Nobel Prize in 1976. GnRH is type of decapeptide that does not contain free amino acids and carboxyl and its molecular structure is: P-Glu-His-Trp-Ser-Tyr-Gly-Leu-Arg-Pro-Gly-NH2, of which the 4th-6th amino acids may form a β turn, in the shape of hairpin, suitable for binding to the receptor, the 2th and 3rd is very important to biological activity, the sixth plays a important role in maintaining the hairpin conformation, and first and the 4th-10th amino acids all are involved in receptor binding, and the replacement of above amino acid residues may lead to loss of vitality or geometric enhancement.

It is easy to be degraded in vivo by proteolytic enzymes, so its half-life period is only 4-8 min. The main action part of its hydrolase peptidase is Gly6-Leu7 and Pro9-Gly10-NH2. To seek for the high efficient and lasting LHRH analogues, more than 3000 kinds of LHRH analogues have been synthesized by picking up or replacing the amino acids in the peptide chain structure. As the half-life period of the synthesized LHRH is long and the effect is stronger, it is more suitable for the treatment of patients than the natural LHRH. The requirement of synthesizing long acting LHRH agonist is to stabilize the molecular structure so that it is not easy to be hydrolyzed by the enzyme, and to increase the binding to the protein in circulation and the cell membrane, and enhance the affinity to the LHRH receptor; for example, the analogues the 6^{th} of which is D-amino acid, and the substituted Gly10 amide group. Such LHRH agonist not only has a greater resistance to protease hydrolysis, but also has a higher affinity for the receptor. The affinity to the receptor can be further enhanced if introducing a large hydrophobic group to the sixth. Such replacement can stabilize the “active” configuration of the releasing hormone analogues and increase the binding with the protein in circulation, thereby prolonging the half-life. The theoretical basis of the early developed the LHRH antagonist was similar to that of the LHRH agonist, which could improve the binding to the receptor, but would produce less readily acceptable side effects of histamine releasing. Therefore, the development of the next generation of LHRH antagonists focuses on the respect of both improving the efficiency and reducing the histamine releasing.

An increasing number of studies have found that the receptors of certain hormones and cytokines have abnormally high expression in the tumor and cancer cells, such as EGFR, LHRHR, etc. Moreover, the molecules hormones and cytokine are relatively small in size, simple in structure, and easy to operate, therefore, as they have great feasibility as the vector of immune toxins. At present, a lot of cytokines and hormones are used as the immune toxin vector, such as IL-2, IL-4, EGF, LHRH, etc., and the expressed recombinant immune toxin proteins all have specific cytotoxic effect. GnRH receptors were first found in the pituitary, and the further study of GnRH and its receptor in recent years, more and more clinical and experimental results show that there is distribution of GnRH and its receptor in peripheral tissue of the pituitary, and the GnRH receptor properties of the normal tissues outside the pituitary and the cancer tissues in its corresponding parts differs much. It is reported that in the literature that the signal transduction pathways mediated by them are different, and it is also reported that the GnRH receptor on the surface of cancer cells may mediate cell apoptosis. In addition, the affinity of the cancer cell membrane surface receptor is usually greater than that of the corresponding normal tissue cell membrane surface receptor. According to reports, three subtypes of LHRH receptor have been found in pituitary and brain cells of the vertebrate bullfrog, and two subtypes have been found in humans, but the distribution and function of them varies from each other; type I receptor binds to type I GnRH, type II receptor binds with type II GnRH, cross reaction between the two is extremely low. A lot of experiments have proved: (1) There is distribution of GnRH receptors on human normal gonadal tissue (including the endometrium, the myometrium, ovary and testis) and the placenta and other cell membranes. (2) There is no existence of GnRH receptor in cell membranes of other normal tissues such as the heart, liver, spleen, lung, kidney, muscles, etc. (3) GnRH receptors are mainly distributed in the membrane of the liver cancer, gastric cancer, pancreatic cancer, colon cancer, ovarian cancer, endometrial cancer, uterine fibroids, breast cancer and prostate cancer cells.

The term “mGnRH” used in this paper refers the matter that can bind to the cells on the surface of which there is GnRH receptor, and can lead protein molecule to act on the target cells to cause cell lesion or death, and the function mechanism of it is equivalent to or higher than that of natural GnRH or its analogues.

Natural GnRH is also known as luteinizing hormone releasing hormone (LHRH or LRH), and it is decapeptide molecule that has the amino acid sequence shown below:
pGlu-His-Trp-Ser-Tyr-Gly-Leu-Arg-Pro-Gly

Since GnRH oriented protein must be added with the promoter and gene enzyme digestion site in gene engineering expression to conduct connection, so in the mGnRH amino acid sequence of the mGnRH-PE38m4a fusion protein in Chinese patent CN200810051112.4, the initiator codon ATG is added, and the correct opening of reading frame is adjusted and GGC codon is added, therefore, the expression product is the dodecapeptide molecules:
Met-Gly-Glu-His-Trp-Ser-Tyr-Trp-Leu-Arg-Pro-Gly

In fusion toxin involved in the invention, the designed directing part is the mutated GnRH amino acid, the sequence of which is:
Glu-His-Trp-Ser-Tyr-Trp-Leu-Arg-Pro-Gly

The amino acid symbols used in this formula are the common three-letter abbreviations for this field, among which the codon of Met-Gly is removed from the N terminal at the time of gene synthesis to facilitate genetic link, and for the sixth amino acids in the new sequence, the mutated Trp is retained at the genetic level, to enhance the binding capacity of the expression product to the corresponding receptor; at the same time, the Escherichia coli preferred codon is introduced into it, to make the expression product suitable to be expressed in Escherichia coli.

Biological activity analysis of the mGnRH-PE38m4a fusion protein we made in accordance with the invention method in Chinese patent CN200810051112.4 has clearly proven that mGnRH-PE38m4a has cytotoxic activity against tumor cells from various organ sources including Hela, but doesn't have that specific cytotoxic activity against normal cells from various tissues, (see embodiment No. 2 of Chinese patent 200810051112.4). In the same time, we have compare the activity of mGnRH-PE38m4a made in accordance with the method of this invention with that of the original products, and the results show that the activity of the sample made in accordance with the this invention patent is better than that of the original products.

The term “IC₅₀” used in this text refers to the concentration of the fusion protein needed to inhibit the protein synthesis of the target cells to achieve 50% of the control group. In this invention, the MTT standard method is used to measure the protein's median inhibitory concentration of in-vitro cultured cells.

The invention further involves the method of using the recombinant DNA technology to produce recombinant fusion protein PE with various protein tandem fusion expression of target specificity and cytotoxicity, and the method includes: (1) provide the expression vector that carries PE38KDEL gene; (2) artificially synthesize the encoding transsulfurase A for molecular series connection and the nucleotide sequence of SUMO enzyme digestion substrate, and clone them into the expression vector of the linear step (1); (3) use the expression vector of step (2) to transform into appropriate host cells; (4) culture the described host cells under the suitable condition for the expression of the of the described fusion protein composed by transsulfurase A, SUMO enzyme digestion substrate and mGnRH-PE38m4a; (5) recover the described fusion protein from the cell cultures.

The PE molecules for the construction of the fusion protein of this invention may be the natural PE molecule the Ia region and most of the Ib region of which are missing, but they can also and are better to be the modified PE38m4a molecules. For example, such modified PE38KDEL molecules may be the PE38m4a the 1^{st}˜4th cysteines (Cys²⁸⁷, Cys³⁷², Cys²⁶⁵ and Cys³⁷⁹) of which were removed or replaced by other amino acids like Lys-Asp-Glu-Leu.

As the molecular weight of the small molecule hormone serving as the directing agent or known as the recognition molecule in the anti tumor chimeric toxin of this invention is relatively small (10 peptides), we can use the universal DNA synthesizer to synthesize the nucleotide sequence which encodes these hormones or their variants on the 1000 A solid phase carrier. In order to facilitate the connection of the PE encoding sequence which is free or carried in the specific recombinant vector, it is practicable to introduce appropriate endonuclease (NcoI for example) enzyme digestion site into the 5′ and/or 3′ terminal of the fusion protein gene sequence, and creates a sticky end suitable for the connection. It is practicable to use the recombinant techniques known to the technical personnel in this field, to clone and encode these synthetic genes whose reading frame is obstructed (or in the form of its cDNA or genomic DNA); in the operation DNA recombination, the standard PCR amplification technique is generally used to amplify the desired gene fragment and cause the appropriate enzyme digestion site. Use restrictive enzyme digestion method to identify the correctness of the sequence link orientation and the possible mutations, and finally identify the sequence by DNA sequencing.

It should be particularly noted that, as relative to the PE38m4a part of the cell toxicity, the molecular weight of the mGnRH in the directing part is much smaller, therefore, the PE38m4a part in the fusion protein produced by both of them is likely to form a new secondary spatial conformation, which may lead to the PE38m4a part's crispation of the mGnRH, thus affecting the receptor binding degree of the fusion protein. However, according to the computer analysis by the technical staff, the crispation of the PE38m4a part doesn't affect the molecular exposure of mGnRH or the binding to the receptor.

The recombinant protein gene will be manipulatively connected to the appropriate expression control sequence, for example, connected to the T₇, trp or the λ, promoter, ribosome binding site and the transcription termination signal suitable for use in Escherichia coli. The known transformation method, such as the calcium chloride treatment method suitable for prokaryotic cell and the electroporation method for mammalian cells, can be used to transform the recombinant plasmid of this invention into the host cell selected. The positive cells to be transformed can be selected based on the antibiotics resistance conferred by the antibiotic resistance gene contained in the plasmid. Once the desired fusion protein is expressed, the fusion protein can be separated and purified according to the method known in this field. For example, centrifugally collect cells from the fermentative cultures and pyrolyze them with lysozyme and ultrasonic wave, then conduct ultra-centrifugation and dose saturated ammonium sulfate in the low concentration of phosphate solution (about 20 mM) for fractional precipitation. Get them going through the ion exchange chromatography (IEC) and IMAC chromatography to purify the desired MGnRH-PE38KDEL recombinant protein.

In general, use polyacrylamide gel to analyze each column chromatography elution part SDS-PAGE with electrophoresis method, and use o polyclonal anti PE antiserum and monitor it with immune blotting method. Carry out tumor cell inhibition tests on the purified products of the recombinant fusion protein to detect the cytotoxicity of the fusion protein (IC₅₀).

The mGnRH-PE38m4a fusion protein of this invention can also be used as the basic active component, and added with one or more pharmaceutically acceptable vectors or excipients, to be made into pharmaceutical compositions suitable for clinical application. The vectors or excipients described include but are not limited to phosphate buffered saline, physiological saline, isotonic glucose solution, glucan, dextran, etc. Depending on the differences in the diseases to treat, one or more of the other natural, synthetic or recombinant active compounds that may have auxiliary function or synergistic effect on the fusion proteins of this invention may be added to the pharmaceutical compositions of this invention. In addition, protein protectant of low molecular weight peptides, glycine or lysine and metal cations (such as Mn²⁺, Mg²⁺, Ca²⁺ and Zn²⁺), as well as stabilizers of polyethylene glycol, carboxymethyl cellulose, poly glycine, glutathione drug can be added to the pharmaceutical compositions of this invention.

The pharmaceutical compositions of this invention can be put into use through the conventional route of administration, especially the extra-gastrointestinal routes, for example drug administration through intravenous, intraperitoneal and intramuscular, intradermal, subcutaneous or mucosal routes. The effective dose range of the pharmaceutical composition of this invention varies from a few nanograms to dozens of mg/kg bodyweight/day, but for each specific patient, the concrete drug dosage will be determined according to the nature and severity of the disease or the pathological state to be treated, the patient's age, weight, the drug reaction ability, the drug administration mode and other factors.

It should be particularly noted that, although the deeper mechanism of action is not clear, our laboratory has demonstrated that the mGnRH-PE38m4a protein of the invention has obvious specificity binding activity and cytotoxicity to tumor cell lines including colon cancer HT-29 cells, ovarian cancer OVCAR3 cells, cervical adenocarcinoma HeLa cells and liver cancer HepG-2 cells.

### DESCRIPTION OF FIGURES

FIG. 1 shows the schematic diagram of the recombinant plasmid used for expressing mGnRH-PE38m4a.

### CONCRETE IMPLEMENTATION METHOD

The following further clarifies the invention with the aid of embodiment, but the technicians in this field realize that these embodiments do not constitute any restriction on the range of the pending claims of the invention.

### Embodiment 1

### Preparation of mGnRH-PE38m4a Fusion Protein

### (1) The Construction and Identification of Recombinant Expression Plasmid

a. Gene synthesis: the expression vector PET-11a-mGnRH-PE38m4a was constructed in embodiment 1 of Chinese patent 200810051112.4, and NdeI incision enzyme is used for connection between the mGnRH and PE38m4a genes, and the invention refers to the publicly acknowledged transsulfurase A and SUMO enzyme recognize substrate sequence, and synthesize the following sequence in vitro:

NcoI endonuclease+ transsulfurase A+HIS6+SUMO enzyme recognition substrate sequence +mGnRH+NdeI endonuclease gene sequence SEQ ID No: 1, and this synthetic sequence forms sticky ends through NcoI and Ndei double enzyme digestion, and insert into the PET-11a-mGnRH-PE38m4a vector of the same enzyme digestion under correct reading frame and in the presence T₄ ligase (Promega), to construct transsulfurase A+HIS6+SUMO enzymes recognition substrate sequence+mGnRH+PE38m4a fusion protein double-stranded expression gene, and the nucleotide sequence of the fusion protein recombinant gene described is SEQ ID NO. 2.

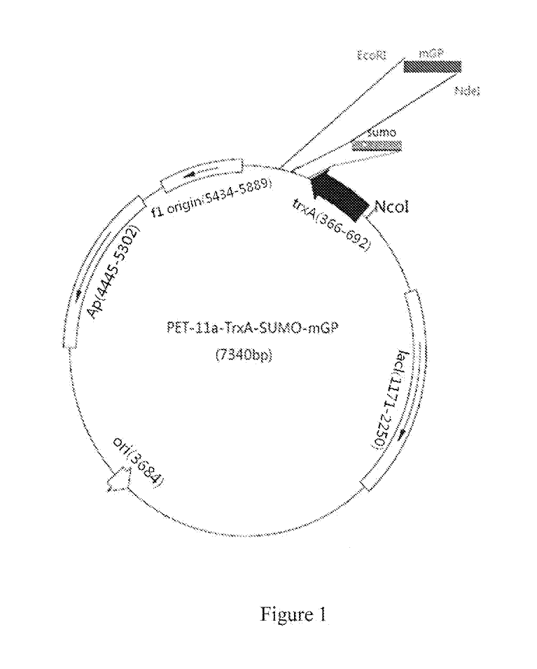

b. The vector constructed transforms the competent Escherichia coli JM105 cells, and culture the transformed cells in ampicillin (50 μg/ml) containing LB culture medium, to amplify the plasmid DNA. After the completion of culture, the cells were crushed, the plasmids are centrifugally collected and DNA sequencing is purified; plasmids with correct sequencing are transformed into Escherichia coli BL21 (DE3) bacterial strains, and enzyme digestion identification method and agarose gel (2%) electrophoresis method are used for identification, and then conduct DNA sequence analysis for the positive recombinant plasmids. FIG. 1 shows the construction of the recombinant plasmid PET-11a-TRXA-SUMO-mGnRH-PE38m4a.

### (2) Expression of the TRXA-SUMO-mGnRH-PE38m4a Fusion Protection and Purification of the Products:

Culture the Escherichia coli BL21(DE3) (containing the T7 RNA polymerase gene) that carries the recombinant gene and is transformed from plasmid in LB agar plate that contains ampicillin (50 μg/ml). After the culturing, choose ampicillin resistant colonies and culture them in ampicillin containing LB culture medium at 37° C. LB (50 UG/ml); when A₆₀₀ reaches about 0.4˜0.6, add 1 mm isopropyl-β-D-galactoside (IPTG) (final concentration of 1 mM), and continue to culture that for 3 hours at 37° C. of, to induce the expression of the target product. Then centrifugally isolate the cells and the culture medium, and add buffer components into the bacterial that contains the target proteins, and the final concentration will reach 50 mm Tris HCl, ph8.0, 1 mm EDTA, ultrasonication. 4° C. centrifugation (20000 g. 30 minutes), take the supernatant (the soluble fraction), that is, the fusion protein crude extracts.

Crude extracts goes through the DEAE-Sepharose Fast Flow column (Pharmacia) of the buffer balancing; use TE (20 mM Tris-HCl, pH8.0.1 mM, EDTA) buffer that contains 0-0.5M NaCl to conduct continuous gradient elution, and collect the peak part of each protein components. After the target component peak part is ultra-filtered, concentrated with medium changed under the action of the small hollow fiber ultra-filter (Milipore) for 30 minutes, make the concentrates go through the XK1.6×10 cm IMAC column (Pharmacia) balanced by 20 mM pH8.0, 0.15M (NaCl) and 20 mM imidazole buffer, and elute it with buffer (20 mM Tris-HCl, pH8.0, 200 mM imidazole) that contains 0.15M NaCl. Collect target protein peak part and dilute it 10 times, and use SUMO for the enzyme digestion of the target protein, 30° C., 4 hr; conduct MAC liquid chromatographic column again in the same conditions, collect the Rukawa peak part and thoroughly dialyze it in 30 mM PBS; after the dialysis, store it in temperature −20° C. for later use, and the purity of the protein SEQ ID No. 3 purified in this way is greater than 97%.

### Embodiment 2

### Use MTT Method to Measure the Effect of the mGnRH-PE38m4a Made by these Two Processes on Hela Cellular Activity

Cell Toxicity Test:

Digest the cultured HeLa monolayer cell with trypsin, and percuss and collect cell suspension, use cell counting plate to count them and adjust the number of cells to 60000/ml, add them into the 96-hole culture plate (5000 cells per hole) in accordance with 80 μl/hole, and culture them for 4 hours under the condition of 5% CO₂, 37° C. Adjust the sample concentration of the mGnRH-PE38m4a protein made by these two processes to 1 mg/ml; Filter and sterilize the quantitative samples, and add different amount of sample into each cell hole in accordance with equal times dilution method; then supplement the culture medium, to enable its total volume to reach 1000, culture it for 12 hours under the condition of 5% CO₂, 37 37° C., add 100 μl MTT staining reagent respectively into each hole of the culture plate, and continue to culture it for 4 hours under the condition of 5% CO₂, 37° C.; measure the light absorption value under 490 nm wavelength, and the concentration (IC50) of the synthesis inhibition of the fusion protein on 50% protein of HeLa tumor cells, and the results are shown in Table 1.

TABLE 1
Comparison of the Products of Two Processes

| Sample Type | 200810051112.4 Process | | The Process of this Invention | |
|---|---|---|---|---|
| Hela Cell IC₅₀ | 0.245 | μg/ml | 0.213 | μg/ml |
| Output | 1.15 | mg | 2.58 | mg |
| (1 gram | | | | |
| bacteria) | | | | |
| Purification | 5 | | 3 | |
| procedures | | | | |
| The cost of | 3.40 | Yuan | 1.05 | Yuan |
| each mg | | | | |

The purification of the samples made by the process of this invention is simple, the operation time is short, the retention of the sample activity is good, and the activity of the samples made by the process of this invention is 15% higher than that of samples made by original process.

What is stated above is the preferred implementation example of this invention, and it should be pointed out that for the ordinary technical personnel in this technology field, on the premise of not divorced from the principles mentioned in this invention, they can also make some improvements and polishing, and these improvements and polishing shall also be regarded as the protection range of the invention.

1. For target specific double-mutant fusion protein, from N-terminal to C-terminal, the fusion protein described includes: the amino acid residues 1-10 of the releasing hormone of gonadotropin the first amino acid of which is glutamine (Glu) and the sixth amino acid of which is modified, and the truncated Pseudomonas aeruginosa exotoxin A amino acid residues, and the optimal amino acid sequence is SEQ ID No. 3.

2. According to the fusion protein described in claim No. 1, the truncated Pseudomonas exotoxin A amino acid residues described are PE40, PE38, PE35, pe23 or the known structure fragments, and they can also be the known structure fragments of PE40, PE38, PE35, PE23 the last four amino acids are mutated into KDEL.

3. According to the fusion protein in claim No. 2, the protein described consists of Met+TrxA+His Tag+Sumo protease recognition sequence+mGnRH+ the mutated PE fragments.

4. According to the fusion protein described in claim No. 1, wherein the modification of the sixth amino acid residue of the releasing hormone of gonadotropin described includes the replacement of the sixth Gly of the of the releasing hormone of gonadotropin by D-Trp or other amino acids.

5. According to the fusion protein in claim No. 4, the sixth Gly of amino acid residue described is acetylated.

6. For fusion protein recombinant gene, its nucleotide sequence is SEQ ID NO. 2.

7. According to the fusion protein in claim No. 1, the amino acid sequence of the fusion protein described is SEQ ID NO. 3.

8. According to the fusion protein described in claim No. 1-6, the chimeric toxin proteins stated is prepared by DNA fusion and recombination technology

9. The application of the fusion protein described in claim No. 1-6 in production of anti tumor drugs.

10. The preparation of the fusion protein described in claim No. 6 includes followings steps:
(1) Construction of the expressive plasmid vector which carries recombinant fusion protein double-strand expression gene, and the nucleotide sequence of the described fusion protein recombinant gene is SEQ ID NO. 2.
(2) The expression of fusion protein: transform the plasmid that carries recombinant gene into Escherichia coli to be expressed therein;
(3) Crush the bacteria and centrifugally extract the supernatant, that is, crude extracts of fusion protein; conduct purification of metal chelating medium in the presence of the imidazole;
(4) Use SUMO protease for enzyme digestion, and make use of the metal chelating medium to isolate and purify the label section and the target protein.

11. According to the preparation method of the fusion protein described in claim No. 10, it is characterized in that: the purification mentioned in step (3) is: the crude extracts go through the DEAE-Sepharose Fast Flow column of the buffer fluid balancing, and use buffer that contains 0-0.5M NaCl to conduct continuous gradient elution, and collect each component peak part of the protein; for the target component peak part, after the hollow fiber ultrahigh purity filter acts on it for 20 to 40 minutes for ultra-filtration, concentration, and medium change, make the concentrates go through the XK1.6×10 cm IMAC column that has been balanced by 20 mM Tris-HCl, pH8.0, 0.15M NaCl, 20 mM imidazole buffer, and elute them with buffer (20 mM Tris-HCl, pH8.0, 200 mM imidazole) that contains 0.15M NaCl, and collect the target protein peak part;

12. According to the preparation method of the fusion protein described in claim No. 11, it is characterized in that: the purification mentioned in step (3) is: dilute the collected target protein peak part ten times, use the SUMO for enzyme digestion of the target protein, 30° C., 4 hr; conduct IMAC liquid chromatography again, collect the Rukawa peak part of the protein into the 30 mM PBS for thorough dialysis; after the dialysis, store it at temperature below −20° C. for later use, thus purified fusion protein is obtained.

13. According to the preparation method of the fusion protein described in claim No. 9, it's characterized by that the structure of the expressive plasmid vector that carries recombinant fusion protein double-strand expression gene is show in FIG. 1.
